SEQUENCE LISTING (60 sequences)

SEQ ID NO: 1 - DNA, 19 bp, Artificial Sequence
  SCNN1A forward primer
gcctgggctgtttctccaa

SEQ ID NO: 2 - DNA, 20 bp, Artificial Sequence
  SCNN1A reverse primer
cgtgagtagccggcagagag

SEQ ID NO: 3 - DNA, 19 bp, Artificial Sequence
  SCNN1B forward primer
gtgcaaagtggccatgagg

SEQ ID NO: 4 - DNA, 21 bp, Artificial Sequence
  SCNN1B reverse primer
gcactggtgaagtttcggaag

SEQ ID NO: 5 - DNA, 20 bp, Artificial Sequence
  SCNN1C forward primer
gccaatcagtgtgcaagcaa

SEQ ID NO: 6 - DNA, 19 bp, Artificial Sequence
  SCNN1C reverse primer
gaagcctcagacggccatt

SEQ ID NO: 7 - DNA, 19 bp, Artificial Sequence
  ACCN2 forward primer
gccaacttccggagcttca

SEQ ID NO: 8 - DNA, 19 bp, Artificial Sequence
  ACCN2 reverse primer
ggcacgagagcagcatgtc

SEQ ID NO: 9 - DNA, 20 bp, Artificial Sequence
  ACCN1 forward primer
catcacagcctgtcggattg

SEQ ID NO: 10 - DNA, 20 bp, Artificial Sequence
  ACCN1 reverse primer
gcaggctctgcacactcctt

SEQ ID NO: 11 - DNA, 19 bp, Artificial Sequence
  ACCN3 forward primer
cccagtccgacctttgaca

SEQ ID NO: 12 - DNA, 20 bp, Artificial Sequence
  ACCN3 reverse primer
tcggcaatccaacaacatgt

SEQ ID NO: 13 - DNA, 21 bp, Artificial Sequence
  ACCN4 forward primer
aacctgcttcccaaccatcac

SEQ ID NO: 14 - DNA, 20 bp, Artificial Sequence
  ACCN4 reverse primer
ctttccccacacagcaccat

SEQ ID NO: 15 - DNA, 20 bp, Artificial Sequence
  ACCN5 forward primer
ctgggctctgtctcgctctt

SEQ ID NO: 16 - DNA, 20 bp, Artificial Sequence
  ACCN5 reverse primer
gttgtcgttggccacatgaa

SEQ ID NO: 17 - DNA, 21 bp, Artificial Sequence
  KCNK2 forward primer
tgccataaggcctctgaatga

SEQ ID NO: 18 - DNA, 21 bp, Artificial Sequence
  KCNK2 reverse primer
ctcagttaggcgaaccctgaa

SEQ ID NO: 19 - DNA, 19 bp, Artificial Sequence
  KCNK10 forward primer
gccgtttcaaggcctcatc

SEQ ID NO: 20 - DNA, 20 bp, Artificial Sequence
  KCNK10 reverse primer
tgagctgttctggcccctta

SEQ ID NO: 21 - DNA, 21 bp, Artificial Sequence
  KCNK4 forward primer
ggagcaagctgaaagccatct

SEQ ID NO: 22 - DNA, 21 bp, Artificial Sequence
  KCNK4 reverse primer
ggctggtaggctggagagttc

SEQ ID NO: 23 - DNA, 21 bp, Artificial Sequence
  TRPM1 forward primer
ctgtccctgtggtggtttgtg

SEQ ID NO: 24 - DNA, 21 bp, Artificial Sequence
  TRPM1 reverse primer
cgtcgcagtatttgtgtgcaa

SEQ ID NO: 25 - DNA, 22 bp, Artificial Sequence
  TRPM2 forward primer
gaggagacacggcagctattct

SEQ ID NO: 26 - DNA, 21 bp, Artificial Sequence
  TRPM2 reverse primer
tgaagtacagggacgccatct

SEQ ID NO: 27 - DNA, 20 bp, Artificial Sequence
  TRPM3 forward primer
tggctgcaggagtactggaa

SEQ ID NO: 28 - DNA, 21 bp, Artificial Sequence
  TRPM3 reverse primer
cctgaagggctggtcttgaag

SEQ ID NO: 29 - DNA, 21 bp, Artificial Sequence
  TRPM4 forward primer
caacaaagtgcatggcaacag

SEQ ID NO: 30 - DNA, 19 bp, Artificial Sequence
  TRPM4 reverse primer
gaattcccggatgaggctg

SEQ ID NO: 31 - DNA, 20 bp, Artificial Sequence
  TRPM5 forward primer
atgggagccagtcctatgca

SEQ ID NO: 32 - DNA, 21 bp, Artificial Sequence
  TRPM5 reverse primer
ccttcacaaacttgcttcgct

SEQ ID NO: 33 - DNA, 20 bp, Artificial Sequence
  TRPM6 forward primer
tctcagccactgagggcaat

SEQ ID NO: 34 - DNA, 21 bp, Artificial Sequence
  TRPM6 reverse primer
ggaagttaatggtgccgaagg

SEQ ID NO: 35 - DNA, 21 bp, Artificial Sequence
  TRPM8 forward primer
ctgctggagtggaaccaactg

SEQ ID NO: 36 - DNA, 20 bp, Artificial Sequence
  TRPM8 reverse primer
agggccgtgaacatgacttc

SEQ ID NO: 37 - DNA, 20 bp, Artificial Sequence
  TRPC1 forward primer
caaagcaacgacaccttcca

SEQ ID NO: 38 - DNA, 20 bp, Artificial Sequence
  TRPC1 reverse primer
gccacatgcgctaaggagaa

SEQ ID NO: 39 - DNA, 21 bp, Artificial Sequence
  TRPC2 forward primer
ccctaccaggagacggagaag

SEQ ID NO: 40 - DNA, 21 bp, Artificial Sequence
  TRPC2 reverse primer
gccaaacatggtccagaagag

SEQ ID NO: 41 - DNA, 21 bp, Artificial Sequence
  TRPC3 forward primer
gggcaggtgacgacttctatg

SEQ ID NO: 42 - DNA, 20 bp, Artificial Sequence
  TRPC3 reverse primer
aggatgatgggcgtgatgtc

SEQ ID NO: 43 - DNA, 20 bp, Artificial Sequence
  TRPC4 forward primer
ccacgaggtccgctgtaact

SEQ ID NO: 44 - DNA, 19 bp, Artificial Sequence
  TRPC4 reverse primer
cgtgagtgcctgaggctgt

SEQ ID NO: 45 - DNA, 20 bp, Artificial Sequence
  TRPC5 forward primer
ccttcgctcatcgccttatc

SEQ ID NO: 46 - DNA, 19 bp, Artificial Sequence
  TRPC5 reverse primer
ccttgagttcccagcccag

SEQ ID NO: 47 - DNA, 21 bp, Artificial Sequence
  TRPC6 forward primer
gcagcagctcctctccatatg

SEQ ID NO: 48 - DNA, 20 bp, Artificial Sequence
  TRPC6 reverse primer
cgaggaccacgaggaatttc

SEQ ID NO: 49 - DNA, 20 bp, Artificial Sequence
  TRPC7 forward primer
tcctggacggagatgctcat

SEQ ID NO: 50 - DNA, 20 bp, Artificial Sequence
  TRPC7 reverse primer
tcctcccagatctccttgca

SEQ ID NO: 51 - DNA, 20 bp, Artificial Sequence
  TRPV1 forward primer
cagcactgcacattgccatt

SEQ ID NO: 52 - DNA, 21 bp, Artificial Sequence
  TRPV1 reverse primer
tccattctccaccaagagggt

SEQ ID NO: 53 - DNA, 20 bp, Artificial Sequence
  TRPV2 forward primer
ggtcatccttcgagacctgc

SEQ ID NO: 54 - DNA, 20 bp, Artificial Sequence
  TRPV2 reverse primer
ggctacagcaaagccgaaaa

SEQ ID NO: 55 - DNA, 20 bp, Artificial Sequence
  TRPV4 forward primer
ctgatgaggagttccgggaa

SEQ ID NO: 56 - DNA, 20 bp, Artificial Sequence
  TRPV4 reverse primer
cgtttcggccattgcttaag

SEQ ID NO: 57 - DNA, 19 bp, Artificial Sequence
  TRPV5 forward primer
gttgcgaatatggcctggg

SEQ ID NO: 58 - DNA, 21 bp, Artificial Sequence
  TRPV5 reverse primer
tcgatacggattctgctcctg

SEQ ID NO: 59 - DNA, 20 bp, Artificial Sequence
  TRPV6 forward primer
tgcagcccaacaaaaccttt

SEQ ID NO: 60 - DNA, 21 bp, Artificial Sequence
  TRPV6 reverse primer
gggcacaagttcaagggactt

(19) United States
(12) Patent Application Publication
(10) Pub. No.: US 2007/0281986 A1
(43) Pub. Date: Dec. 6, 2007

(54) Methods and Compositions for Modulating Satiety

(76) Inventors: Gregory Royce Collier, Barwon Heads Victoria (AU); Kenneth Russell Walder, Ocean Grove Victoria (AU)

Correspondence Address: THE MCCALLUM LAW FIRM, P. C., 685 BRIGGS STREET, PO BOX 929, ERIE, CO, 80516, US

(21) Appl. No.: 10/588,576

(22) PCT Filed: Feb. 1, 2005
(86) PCT No.: PCT/AU05/00120

§ 371 Date: May 24, 2007

Related U.S. Application Data
(60) Provisional application No. 60/541,862, filed on Feb. 3, 2004.
(60) Provisional application No. 60/592,052, filed on Jul. 28, 2004.

(51) Int. Cl. A61K 31/4178

(52) U.S. Cl. 514/403

(57) ABSTRACT

The present invention relates generally to a method for modulating the sensation of satiety perception and to agents useful for same. More particularly, the present invention identifies molecules which interact with ion channels thereby modulating the perception of satiety. The present invention contemplates, therefore, a method for modulating gastric distension by the administration of agents which control the activation or inhibition of mechanoreceptors. The present invention further provides compositions comprising these agents and methods of treatment using same. In a particular embodiment, the present invention permits modulation of the sensation of gastric distension by the administration of agents which control the activation of mechanoreceptors associated with mechanical stretch.

### BACKGROUND OF THE INVENTION

1. Field of the Invention

The present invention relates generally to a method for modulating the sensation of satiety perception and to agents useful for same. Such agents are useful in modulating, controlling or otherwise affecting inter alia obesity, anorexia, weight maintenance, metabolic energy levels and/or inflammatory conditions in a subject. More particularly, the present invention identifies molecules which interact with ion channels thereby modulating the perception of satiety. The present invention contemplates, therefore, a method for modulating the perception of gastric distension by the administration of agents which control the activation or inhibition of mechanoreceptors. The present invention further provides compositions comprising these agents and methods of treatment using same. In a particular embodiment, the present invention permits modulation of perception of gastric distension by the administration of agents which control the activation of mechanoreceptors associated with mechanical stretch.

2. Description of the Prior Art

Bibliographic details of references in the subject specification are also listed at the end of the specification.

Reference to any prior art in this specification is not, and should not be taken as, an acknowledgment or any form of suggestion that this prior art forms part of the common general knowledge in any country.

Obesity is defined as a pathological excess of body fat and is the result of an imbalance between energy intake and energy expenditure for a sustained period of time. Obesity is the most common metabolic disease found in affluent societies. The prevalence of obesity in these affluent societies is alarmingly high, ranging from 10% to upwards of 50% in some sub-populations (Bouchard, The Genetics of Obesity, Boca Raton: CRC Press, 1994). Of particular concern is the fact that the prevalence of obesity appears to be rising consistently in affluent societies and is now increasing rapidly in less prosperous nations as they become more affluent and/or adopt cultural practices similar to those in more affluent countries (Zimmet, Diabetes Care 15: 232-252, 1992). The escalating rates of obesity globally have resulted in the World Health Organisation declaring an obesity epidemic worldwide (World Trade Organisation. Obesity. Preventing and managing the global epidemic. Report of a WHO Consultation on Obesity. Geneva: World Health Organisation, 1998).

In Australia, an AusDiab study estimated that 7.5 million Australians (60%) aged 25 years and over were overweight or obese. Of these, 2.6 million (21%) were obese (BMI>30) (Dunstan et al., Diabetes Res. Clin. Pract. 57: 119-129, 2002). Similarly, the prevalence of obesity in the U.S. increased substantially between 1991 and 1998, increasing from 12% to 18% in Americans during this period (Mokdad et al., JAMA 282(16): 1519-1522, 1999).

The high and increasing prevalence of obesity has serious health implications for both individuals and society as a whole. Obesity is a complex and heterogeneous disorder and has been identified as a key risk indicator of preventable morbidity and mortality. Obesity, for example, increases the risk of a number of other metabolic conditions including Type 2 diabetes and cardiovascular disease (Must et al., JAMA 282(16): 1523-1529, 1999, Kopelman, Nature 404: 635-643, 2000). Alongside obesity the prevalence of diabetes continues to increase rapidly. The AusDiab survey referred to above estimated that close to 1 million Australians aged 25 years and over have Type 2 diabetes (Dunstan et al., 2002 supra). This represents approximately 7.5% of the population. In the U.S., the number of adults with diabetes increased by 49% between 1991 and 2000 (Marx, Science 686-689, 2002). It has been estimated that about 17 million people in the U.S. have Type 2 diabetes and an equal number are thought to be pre-diabetic (Marx, 2002, supra). In Australia, the annual costs of obesity associated with diabetes and other disease conditions has been conservatively estimated to be AUS $810 million for 1992-93 (National Health and Medical Research Council, Acting on Australia's weight: A strategy for the prevention of overweight and obesity. Canberra: National Health and Medical Research Council, 1996). The direct costs of diabetes and its complications in Australia in 1993-94 were estimated at $681 million, or 2.2% of total health system costs in that year (Australian Institute of Health and Welfare (AIWH), Australia's Health, 2002, Canberra: AIWH).

Obesity also has both metabolic and physiological bases and these need to be understood in development of therapeutic and prophylactic regimes. There is a need, therefore to investigate other physiological influences of obesity and other conditions.

The role of physiological gastric distension in the control of short-term satiety has been known for some time, and is mediated by both peptides secreted from the gastrointestinal tract in response to distension, as well as the enteric nervous system. Studies in rats have demonstrated that vagal afferent fibres respond to gastric distension, and mediate part of the physiological response (Phillips and Powley, Am J Physiol 274: R1626-R1638, 1998; Ozaki, et al. J Neurophysiol 82: 2210-2220, 1999; Ozaki and Gebhart, Am J Physiol 281: G1449-G1459, 2001). Further investigations demonstrated that most of the vagal nerve endings in the stomach that mediate the detection of distension have unique structural characteristics, and are known as Intraganglionic Laminar Endings (IGLEs) and Intramuscular Arrays (IMAs). These vagal nerve endings are responsive to gastric distension and play a role in short term satiety (Berthoud, et al. Am J Physiol 280: R1371-R1381, 2001; Fox, et al. J Neuroscience 21: 8602-8615, 2001; Zagorodnyuk, et al. J Physiol 534: 255-268, 2001). However, the molecular mechanisms by which the physical stimulus of distension is converted into a chemical/electrical signal (a process known as mechanotransduction), and subsequent physiological adaptation, are unclear.

In accordance with the present invention, it has been surprisingly determined that modulators of calcium flux across the cell membrane are useful in the treatment or prevention or modulation or control of obesity, anorexia, satiation, weight maintenance, metabolic energy levels and inflammatory conditions.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated, in part, upon the identification of specific molecules which interact with mechanoreceptors independent of mechanical stimulus or their functional, structural or evolutionary equivalents or homologues including polymorphic variants and modulate the perception of satiety. Such molecules are important in modulating the sensation of satiety perception of a subject. Specifically, mechanoreceptors are identified which are involved in gastric distension. In accordance with one embodiment of the present invention, agents which modulate the activity of these receptors are proposed to modulate a subject's perception of satiety. These agents enhance or suppress the function of the mechanoreceptors including the activation of these mechanoreceptors and alter the physical sensations associated with these receptors.

The present invention, therefore, identifies a family of mechanoreceptors as targets for the modulation of the perception of satiety and agents which are useful in modulating their activation. The present invention enables rational drug design or screening of natural products or chemical libraries for agents which modulate the perception of satiety by modulating the activation of mechanoreceptors or their functional, structural or evolutionary equivalents or homologues. In accordance with the present invention it is proposed that physiological gastric distension can either increase or decrease the expression of these putative mechanoreceptors. Consequently, it is proposed that either enhancing or reducing the activation of these mechanoreceptors can alter the sensation of satiety.

In a preferred aspect, the mechanoreceptors are ion channels, and preferably from the families of ion channels selected from EnaCs, ASICs, mechanosensitive potassium channels and transient receptor potential ion channels.

Even more preferably, the ion channels are selected from the group consisting of ENAC, βENAC, γENAC, ACCN3, ACCN4, ASIC1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV1, TRPV2, TRPV3, TRPV6 and TRPM8. In addition, mechanoreceptors may comprise homo- or hetero-multimeric complexes of one or more of these ion channels.

The present invention also describes, in a preferred embodiment, the use of modulators of calcium flux across the cell membrane to modulate, control or otherwise affect obesity, anorexia, satiation, weight maintenance, metabolic energy levels and inflammatory conditions. By “calcium flux” is included “calcium uptake” or “calcium release” which includes the movement of calcium ions into or out of cells in the stomach wall including neuronal cells of the myenteric plexus. Such cells are particularly important for signalling a level of sensation of satiation through the vagus nerve to the brain.

The modulatory agents of the present invention may be chemical agents such as synthetic or recombinant molecules, polypeptides, peptides or proteins, lipids, glycoproteins or other naturally or non-naturally occurring molecules or analogs thereof. Alternatively, genetic agents such as DNA (gDNA, cDNA), RNA (sense RNAs, antisense RNAs, mRNAs, tRNAs, rRNAs, small interfering RNAs (siRNAs), micro RNAs (miRNAs), small nucleolar RNAs (snoRNAs, small nuclear (snRNAs)) ribozymes, aptamers, DNAzymes or other ribonuclease-type complexes may be employed.

Preferred modulators of calcium flux across the cell membrane are selected from a blocker or promoter of mechanoreceptor TRPV2 calcium channels, a biological dye which inhibits or promotes calcium uptake and salts, homologs, orthologs, analogs, isomers, derivatives or functional equivalents thereof. Most preferred compounds are selected from 1-[β-[3-(4-methoxyphenyl)propoxy]-4-methoxyphenethyl]-1H-imidazole and a ruthenium red dye or salts, homologs, orthologs, analogs, isomers, enantiomers, derivatives or functional equivalents thereof as well as pharmaceutical compositions comprising same. Particularly preferred ruthenium dyes include ruthenium(6+), tetradecaamminedi-m-oxotri-, hexachloride, trans-(8Cl) or a stereoisomer or enantiomer thereof and/or ammoniated ruthenium oxychloride or a stereoisomer or enantiomer thereof. All such modulators of calcium flux across the cell membrane are referred to as inter alia “therapeutic” agents, compounds, medicaments or molecules. Notwithstanding that the preferred compounds block TRPV2 calcium channels, this may not necessarily be the mode of action.

The chemical or genetic agents may be formulated into a range of compositions.

In a preferred aspect, the mechanoreceptors of the present invention are involved with the sensation of gastric distension. Accordingly, agents of the present invention which enhance or inhibit the activation of these gastric mechanoreceptors alter the perception of satiety and hence change a subject's desire to consume orally.

The present invention further contemplates a method for the prophylaxis or treatment of a condition including a condition characterized in part by the presence of a symptom associated with a disorder or disease associated with obesity, anorexia, need for satiation, weight maintenance, metabolic energy levels or inflammation in a subject, the method comprising the administration of a therapeutic agent selected from a calcium uptake inhibitor or promoter, a blocker or promoter of TRPV2 calcium channels and a biological dye which inhibits or promotes calcium uptake.

The compositions of the present invention may be used to treat animals including avian and mammalian animals such as human subjects with a range of conditions associated with gastric disorders. In particular, gastric disorders associated with either an excessive or inadequate dietary intact. These gastric disorders include, without being limited to, obesity, anorexia, bulimia, diabetes and/or energy imbalance, sleep apnoea, neural injury, neurological diseases, inflammation, severe burns, severe trauma, chronic non-neurological diseases, chronic infections, chronic corticosteroid administration, AIDS, and the like. Neural injuries, which impact on a subject's dietary intake, include acute brain injuries, traumatic brain injuries, closed head injuries, stroke, and the like. Neurological diseases include chronic neurodegenerative diseases such as Alzheimer's disease, Parkinson's disease, Huntington's disease, multiple sclerosis, and the like.

A summary of the sequence identifiers used throughout the subject specification is provided in Table 1.

TABLE 1
Summary of Sequence Identifers

| SEQUENCE ID NO: | DESCRIPTION |
|---|---|
| SEQ ID NO: 1 | Forward Primer SCNN1A |
| SEQ ID NO: 2 | Reverse Primer SCNN1A |
| SEQ ID NO: 3 | Forward Primer SCNN1B |
| SEQ ID NO: 4 | Reverse Primer SCNN1B |
| SEQ ID NO: 5 | Forward Primer SCNN1C |
| SEQ ID NO: 6 | Reverse Primer SCNN1C |
| SEQ ID NO: 7 | Forward Primer ACCN2 |
| SEQ ID NO: 8 | Reverse Primer ACCN2 |
| SEQ ID NO: 9 | Forward Primer ACCN1 |
| SEQ ID NO: 10 | Reverse Primer ACCN1 |
| SEQ ID NO: 11 | Forward Primer ACCN3 |
| SEQ ID NO: 12 | Reverse Primer ACCN3 |
| SEQ ID NO: 13 | Forward Primer ACCN4 |
| SEQ ID NO: 14 | Reverse Primer ACCN4 |
| SEQ ID NO: 15 | Forward Primer ACCN5 |
| SEQ ID NO: 16 | Reverse Primer ACCN5 |
| SEQ ID NO: 17 | Forward Primer KCNK2 |
| SEQ ID NO: 18 | Reverse Primer KCNK2 |
| SEQ ID NO: 19 | Forward Primer KCNK10 |
| SEQ ID NO: 20 | Reverse Primer KCNK10 |
| SEQ ID NO: 21 | Forward Primer KCNK4 |
| SEQ ID NO: 22 | Reverse Primer KCNK4 |
| SEQ ID NO: 23 | Forward Primer TRPM1 |
| SEQ ID NO: 24 | Reverse Primer TRPM1 |
| SEQ ID NO: 25 | Forward Primer TRPM2 |
| SEQ ID NO: 26 | Reverse Primer TRPM2 |
| SEQ ID NO: 27 | Forward Primer TRPM3 |
| SEQ ID NO: 28 | Reverse Primer TRPM3 |
| SEQ ID NO: 29 | Forward Primer TRPM4 |
| SEQ ID NO: 30 | Reverse Primer TRPM4 |
| SEQ ID NO: 31 | Forward Primer TRPM5 |
| SEQ ID NO: 32 | Reverse Primer TRPM5 |
| SEQ ID NO: 33 | Forward Primer TRPM6 |
| SEQ ID NO: 34 | Reverse Primer TRPM6 |
| SEQ ID NO: 35 | Forward Primer TRPM8 |
| SEQ ID NO: 36 | Reverse Primer TRPM8 |
| SEQ ID NO: 37 | Forward Primer TRPC1 |
| SEQ ID NO: 38 | Reverse Primer TRPC1 |
| SEQ ID NO: 39 | Forward Primer TRPC2 |
| SEQ ID NO: 40 | Reverse Primer TRPC2 |
| SEQ ID NO: 41 | Forward Primer TRPC3 |
| SEQ ID NO: 42 | Reverse Primer TRPC3 |
| SEQ ID NO: 43 | Forward Primer TRPC4 |
| SEQ ID NO: 44 | Reverse Primer TRPC4 |
| SEQ ID NO: 45 | Forward Primer TRPC5 |
| SEQ ID NO: 46 | Reverse Primer TRPC5 |
| SEQ ID NO: 47 | Forward Primer TRPC6 |
| SEQ ID NO: 48 | Reverse Primer TRPC6 |
| SEQ ID NO: 49 | Forward Primer TRPC7 |
| SEQ ID NO: 50 | Reverse Primer TRPC7 |
| SEQ ID NO: 51 | Forward Primer TRPV1 |
| SEQ ID NO: 52 | Reverse Primer TRPV1 |
| SEQ ID NO: 53 | Forward Primer TRPV2 |
| SEQ ID NO: 54 | Reverse Primer TRPV2 |
| SEQ ID NO: 55 | Forward Primer TRPV4 |
| SEQ ID NO: 56 | Reverse Primer TRPV4 |
| SEQ ID NO: 57 | Forward Primer TRPV5 |
| SEQ ID NO: 58 | Reverse Primer TRPV5 |
| SEQ ID NO: 59 | Forward Primer TRPV6 |
| SEQ ID NO: 60 | Reverse Primer TRPV6 |

### DETAILED DESCRIPTION OF THE PREFERRED EMBODIMENTS

The terminology used herein to describe the subject invention is for the purpose of describing particular embodiments and is not necessarily intended to be limiting.

The present invention provides a method for modulating the perception of satiety in a subject the method comprising administering to the subject an effective amount of an agent which modulates the activity or function of a mechanoreceptor or a functional, structural or evolutionary equivalent or homolog including polymorphic variant thereof. The method of the present invention is useful for the treatment or prophylaxis of a range of conditions including anorexia, weight maintenance, metabolic energy levels and/or inflammatory conditions.

Reference to a “mechanoreceptor” encompasses any or all receptors involved in mechanotransduction including functional, structural or evolutionary equivalents or homologs including polymorphic variants, from any particular species. Human forms of mechanoreceptors are especially contemplated for use in developing agents for use in human subjects. However, the present invention also has application in the veterinary, agricultural and wild life animal industries.

As used herein, the term “mechanoreceptor” refers to a specialised sensory end organ that responds to mechanical stimuli such as tension, pressure or displacement. Types of mechanoreceptors include ion channels including EnaCs, ASICs, mechanosensitive potassium channels, transient receptor potential ion channels, muscle spindles and tendon organs. Reference to mechanoreceptors encompasses any or all ion channels which are involved in the process of mechanotransduction. Calcium ion channels are particularly preferred such as but not limited to TRPV1 through TRPV6 inclusive and in particular TRPV2.

Although the present invention is particularly directed to the perception of satiety with regard to dietary requirements, homologous mechanoreceptors may be involved in perception of satiety in relation to other forms of physical stimuli and the modulation of those mechanoreceptors to mimic levels of satiety is also contemplated by the present invention.

As used herein, reference to an “ion channel” includes any transmembrane protein or protein complex or non-proteinaceous component that forms or can be activated or induced to form a channel through which specific inorganic ions diffuse. Ion channel activation can occur via a membrane potential, drug, transmitter, cytoplasmic messenger, or a mechanical deformation or stretch. Activation or inactivation of an ion channel via a specific ligand can either occur via the ligand binding directly to a protein component of the ion channel (i.e. direct activation or inhibition) or may occur via the binding of a ligand of the mechanoreceptor, mechanoreceptor region or mechanoreceptor complex or a proteinaceous or non-proteinaceous component of the mechanoreceptor, mechanoreceptor region or mechanoreceptor complex (i.e. indirect activation or inhibition). Ion channels may be activated by both mechanical deformation or via the binding of a specific ligand resulting in mechanical deformation including conformational deformation or chemical or electrical stimulus. Ion channels of the present invention are preferably activated by a mechanical deformation.

Mechanoreceptors or ion channels of the specific invention are any mechanoreceptor or ion channel, whose function, when altered, involves an alteration of the perception of satiety which would otherwise have occurred in response to a physical stimulus or a desire for a physical stimulus. In one aspect, the mechanoreceptors of the present invention are those mechanoreceptors or ion channels associated with modulating the sensation of gastric distension. In a preferred aspect, the mechanoreceptors or ion channels of the present invention are selected from ENAC, βENAC, γENAC, ACCN3, ACCN4, ASIC1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV1, TRPV2, TRPV3, TRPV6 and TRPM8 or a component or combination thereof.

Accordingly, one aspect of the present invention contemplates a method for modulating the perception of satiety in a subject, said method comprising administering to said subject an effective amount of an agent selected from the list consisting of:
(i) an agent which is an agonist of a mechanoreceptor selected from the list consisting of ENAC, βENAC, γENAC, ACCN3, ACCN4, ASIC1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV1, TRPV2, TRPV3, TRPV6 and TRPM8;
(ii) an agent which is an antagonist of a mechanoreceptor list in (i);
(iii) an agent which inhibits expression of a gene encoding a mechanoreceptor listed in (i); and
(iv) an agent enhance expression of a gene encoding a mechanoreceptor listed in (i);
   wherein increasing or decreasing the level of or activity of the mechanoreceptors changes the perception of satiety in said subject.

The phrase “modulating the sensation of satiety or perception of satiety” refers broadly to altering a subject's state of being satisfactorily full and not wanting to take more. This state may be in reference to any sense, and can involve any part of a subject. The phrase specifically encompasses altering the level of satiety to a specific sense in a subject. In a preferred embodiment sensation of satiety is associated with the gastric system and refers to a subject's dietary intake.

It must be noted that, as used in the subject specification, the singular forms “a”, “an” and “the” include plural aspects unless the context clearly dictates otherwise. Thus, for example, reference to “an agent” includes a single agent, as well as two or more agents; reference to “a mechanoreceptor” includes a single mechanoreceptor, as well as two or more mechanoreceptors; and so forth.

Reference to modulating the “sensation of satiety perception or perception of satiety” is meant in its broadest sense and encompasses agents which directly enhance or diminish the perception of satiety by either increasing or decreasing or blocking the activation of mechanoreceptors. Of the latter agents, genetic silencing agents such as to small interfering RNAs or ribozymes which reduce or inhibit ligand availability, dominant negative mutants or parts of the ligand which affect binding but not functional activity are contemplated, among others. The functional consequences of modulating mechanoreceptor activation using an effective amount of an agent is to alter the perception of a particular sensation without supplying mechanical pressure or distortion.

The term “effective amount” of an agent means a sufficient amount of the agent to provide the desired therapeutic or physiological effect. Thus, an “effective amount” of an agent includes a sufficient amount of the agent to modulate the sensation of satiety perception in a subject. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

Agents of the present invention encompass compounds which selectively bind to a mechanoreceptor, either enhancing, preventing or diminishing the function of the mechanoreceptor, including its activation. The agents of the present invention may function in a variety of ways. These include, without being limited to, agents which cover or block the channel of the mechanoreceptor or ion channel preventing the transfer of ions across the cell membrane. In addition, these agents bind to a compartment at or proximal to the site of the mechanoreceptor which results in a conformational alteration to the membrane or the mechanoreceptor complex itself. Alternatively or as well as, the agents may bind to a component of the mechanoreceptor or ion channel thereby influencing its ability to participate in the functioning of the mechanoreceptor complex. Components of the mechanoreceptor may be proteinaceous, non-proteinaceous, lipids or carbohydrates. Conversely, the agents of the invention may bind to a ligand separate from the mechanoreceptor or ion channel, and as such either enhance, prevent or diminish the function of the mechanoreceptor, including its activation, directly or indirectly.

One form of an agent useful in the practice of the present invention is derived from a molecule which interacts with the mechanoreceptor or component thereof and in particular a naturally occurring endogenous ligand. In a particular embodiment where the ligand is a proteinaceous or non-proteinaceous molecule the agent may be a part or derivative of that ligand. A fragment of a proteinaceous ligand is contemplated comprising from about 5 to at least about 30 contiguous amino acids. With respect to a non-proteinaceous, carbohydrate based ligand, such as a GAG or a polyunsaturated fatty acid, the agent may comprise a derivative of these molecules comprising up to less than about 10% the full length molecule.

For example, proteinaceous agents of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of an amino acid sequence of the invention with proteinases such as endoLys-C, endoArg-C, endoGlu-C and staphylococcus V8-protease. The digested fragments can be purified by, for example, high performance liquid chromatographic (HPLC) techniques. Any such fragment, irrespective of its means of generation, is to be understood as being encompassed by the term “derivative” as used herein.

Thus proteinaceous derivatives, or the singular proteinaceous derivative, encompass parts, mutants, homologs, fragments, analogues as well as hybrid or fusion molecules and glycosylaton variants. Derivatives also include molecules having a percent amino acid sequence similarity over a window of comparison after optimal alignment with the naturally occurring molecule with a difference of at least 1%.

Another form of an agent comprises the naturally occurring molecule having a chemical modification. Such a molecule is referred to herein as an analog. Analogs contemplated herein include but are not limited to modification to side chains, incorporating of unnatural amino acids and/or their derivatives during peptide, polypeptide or protein synthesis and the use of crosslinkers and other methods which impose conformational constraints on the proteinaceous molecule or their analogs. This term also does not exclude modifications of the polypeptide, for example, glycosylations, acetylations, phosphorylations and the like. Included within the definition are, for example, polypeptides containing one or more analogs of an amino acid (including, for example, unnatural amino acids such as those given in Table 2) or polypeptides with substituted linkages. Such polypeptides may need to be able to enter the cell.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid, contemplated herein is shown in Table 2.

TABLE 2
Codes for non-conventional amino acids

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-Nmethylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2- | Nmbc | | |
| diphenyl-ethylamino)cyclopropane | | | |

Crosslinkers can be used, for example, to stabilize 3D conformations, using homobifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogs by introducing covalent bonds such as forming an amide bond between the N and C termini, between two side chains or between a side chain and the N or C terminus.

Mimetics are another useful group of compounds. The term is intended to refer to a substance which has some chemical similarity to the molecule it mimics, but which enhances or diminishes or prevents the activation of a mechanoreceptor. A peptide mimetic may be a peptide-containing molecule that mimics elements of protein secondary structure (Johnson et al., “Peptide Turn Mimetics” in Biotechnology and Pharmacy, Pezzuto et al., Eds., Chapman and Hall, New York, 1993). The underlying rationale behind the use of peptide mimetics is that the peptide backbone of proteins exists chiefly to orient amino acid side chains in such a way as to facilitate molecular interactions such as those of antibody and antigen, enzyme and substrate or scaffolding proteins. A peptide mimetic is designed to permit molecular interactions similar to the natural molecule. Peptide or non-peptide mimetics may be useful, for example by up-regulating the activation of a mechanoreceptor and thereby increase the perception of satiety. Alternatively, the peptide or non-peptide mimetics may be useful in down-regulating or suppressing the activation of a mechanoreceptor, thereby decreasing the sensation of satiety perception.

The designing of mimetics to a pharmaceutically active compound is a known approach to the development of pharmaceuticals based on a “lead” compound. This might be desirable where the active compound is difficult or expensive to synthesize or where it is unsuitable for a particular method of administration, e.g. peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g. by substituting each residue in turn. Alanine scans of peptides are commonly used to refine such peptide motifs. These parts or residues constituting the active region of the compound are known as its “pharmacophore”.

Once the pharmacophore has been found, its structure is modelled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modelling process.

In a variant of this approach, the three-dimensional structure of the receptor and its binding partner are modelled. This can be especially useful where the receptor and/or binding partner change conformation on binding, allowing the model to take account of this in the design of the mimetic. Modelling can be used to generate inhibitors which interact with the linear sequence or a three-dimensional configuration.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

The goal of rational drug design is to produce structural analogs of biologically active polypeptides of interest or of small molecules with which they interact (e.g. agonists, antagonists, inhibitors or enhancers) in order to fashion drugs which are, for example, more active or stable forms of the polypeptide, or which, e.g. enhance or interfere with the function of a polypeptide in vivo. See, e.g. Hodgson (Bio/Technology 9: 19-21, 1991). In one approach, one first determines the three-dimensional structure of a protein of interest by x-ray crystallography, by computer modelling or most typically, by a combination of approaches. Useful information regarding the structure of a polypeptide may also be gained by modelling based on the structure of homologous proteins. An example of rational drug design is the development of HIV protease inhibitors (Erickson et al., Science 249: 527-533, 1990).

Proteinaceous agents of the present invention may be conveniently prepared by modifying the nucleotide sequence of a genetic molecule encoding a naturally occurring mechanoreceptor ligand. In relation to genetic molecules, the terms mutant, part, derivative, homolog, analog or mimetic have, mutatis mutandis, analogous meanings to the meanings ascribed to these forms in relation to proteinaceous molecules.

A nucleic acid encoding of a naturally occurring ligand is conveniently defined as the naturally occurring nucleotide sequence with a single or multiple nucleotide substitution, deletion or addition.

The terms “similarity” or “identity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (Nucl. Acids Res. 25: 3389, 1997). A detailed discussion of sequence analysis can be found in Unit 19.3 of Ausubel et al. (“Current Protocols in Molecular Biology” John Wiley & Sons Inc, 1994-1998, Chapter 15).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Derivatives of naturally occurring genetic molecules may also be defined as being encoded by a nucleic acid molecule which is capable of hybridizing to a reference sequence or a complementary form thereof under low stringency conditions.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C) % (Marmur and Doty, J. Mol. Biol. 5: 109, 1962). However, the Tₘ of a duplex nucleic acid molecule decreases by 1° C. with every increase of 1% in the number of mismatch base pairs (Bonner and Laskey, Eur. J. Biochem. 46: 83, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

As indicated above, agents useful in the practice of the present invention may also comprise nucleic acid molecules or modified forms thereof. Examples of such nucleic acid molecules include DNA (genomic, cDNA), RNA (sense RNAs, antisense RNAs, mRNAs, tRNAs, rRNAs, small interfering RNAs (SiRNAs), micro RNAs (miRNAs), small nucleolar RNAs (SnoRNAs), small nuclear (SnRNAs)) ribozymes, aptamers, DNAzymes or other ribonuclease-type complexes. Other nucleic acid molecules will comprise promoters or enhancers or other regulatory regions which modulate transcription. The aim of these molecules is to modulate the levels of components involved in the mechanoreceptor complex.

Accordingly, the present invention extends to a genetic approach for modulating the perception of satiety using nucleic acid constructs which modulate the levels of proteinaceous components.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, alkylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Antisense polynucleotide sequences, for example, are useful in silencing transcripts. Furthermore, polynucleotide vectors containing all or a portion of a mechanoreceptor gene locus or molecule which binds to a mechanoreceptor may be placed under the control of a promoter in an antisense orientation and introduced into a cell. Expression of such an antisense construct within a cell will interfere with target transcription and/or translation. Furthermore, co-suppression and mechanisms to induce RNAi or siRNA may also be employed. Alternatively, antisense or sense molecules may be directly administered. In this latter embodiment, the antisense or sense molecules may be formulated in a composition and then administered by any number of means to target cells.

A variation on antisense and sense molecules involves the use of morpholinos, which are oligonucleotides composed of morpholine nucleotide derivatives and phosphorodiamidate linkages (for example, Summerton and Weller, Antisense and Nucleic Acid Drug Development 7: 187-195, 1997). Such compounds are injected into embryos and the effect of interference with mRNA is observed.

In one embodiment, the present invention employs compounds such as oligonucleotides and similar species for use in modulating the activation of a mechanoreceptor. This is accomplished by providing oligonucleotides which specifically hybridize with one or more mechanoreceptors. The oligonucleotides may be provided directly to a cell or generated within the cell. As used herein, the terms “target nucleic acid” and “nucleic acid molecule encoding an inhibitor” have been used for convenience to encompass DNA encoding the inhibitor, RNA (including pre-mRNA and mRNA or portions thereof) transcribed from such DNA, and also cDNA derived from such RNA. The hybridization of a compound of the subject invention with its target nucleic acid is generally referred to as “antisense”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In one example, the result of such interference with target nucleic acid function in modulation of the activation of a mechanoreceptor. In the context of the present invention, “modulation” and “modulation of activation” mean either an increase (enhanced) or a decrease (inhibition) in the activation of an mechanoreceptor.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e. under physiological conditions in the case of in vivo assays or therapeutic treatment, and under conditions in which assays are performed in the case of in vitro assays.

“Complementary” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds may be introduced in the form of single-stranded, double-stranded, circular or hairpin oligomeric compounds and may contain structural elements such as internal or terminal bulges or loops. Once introduced to a system, the compounds of the invention may elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals.

While oligonucleotides are a preferred form of the compounds of this invention, the present invention contemplates other families of compounds as well, including but not limited to oligonucleotides, analogs and mimetics such as those herein described.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds may have internal nucleobase complementarity and may, therefore, fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Specific examples of antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

In a further aspect of the present invention, the agents which interact with the mechanoreceptor to promote or disrupt activation of the mechanoreceptor and modulate satiety.

In another embodiment, the agent enhances activation of the mechanoreceptor and alters the perception of satiety.

In another embodiment, the agent decreases or prevents activation of the mechanoreceptor and alters the perception of satiety.

Reference to a cell herein includes any cell which expresses mechanoreceptors. The mechanoreceptors may either be endogenously produced or the cell may be genetically manipulated to produce mechanoreceptors. In preferred embodiments, the mechanoreceptors of the present invention are related to responses associated with gastric distension.

In a further aspect of this embodiment, the agent essentially comprises all or part of the sequence of amino acids forming the activation portion of a mechanoreceptor or a molecule which binds to the activation portion of a mechanoreceptor.

In a further aspect of the present invention, the agents which modulate activation of a mechanoreceptor and/or enhance or decrease or prevent activation of a mechanoreceptor are agents which increase or decrease the perception of satiety.

In a further embodiment, the present invention provides a method for modulating the sensation of satiety perception in a subject comprising administering to the subject an agent which either increases or decreases or prevents activation of a mechanoreceptor.

In a preferred aspect, the present invention provides methods for enhancing the perception of satiety and thereby decreasing a subject's desire to eat. In a related aspect, the present invention provides methods for suppressing the sensation of satiety perception and thereby increasing the desire of a subject to eat.

Screening assays for establishing the effects of different agents are well known to those of skill in the art and include such assays as FRET and FLIPR and patch-clamp and voltage-clamp, all of which screen the function of an ion channel.

Fluorescence resonance energy transfer (FRET) is a distance-dependent interaction between the electronic excited states of two dye molecules in which excitation is transferred from a donor molecule to an acceptor molecule without emission of a photon. The efficiency of FRET is dependent on the inverse sixth power of the intermolecular separation, making it useful over distances comparable with the dimensions of biological macromolecules. Thus, FRET is an important technique for investigating a variety of biological phenomena that produce changes in molecular proximity. When FRET is used as a contrast mechanism, colocalization of proteins and other molecules can be imaged with spatial resolution beyond the limits of conventional optical microscopy (Gonzalez et al. DDT 4(9):431-439, 1999).

Fluorescence Imaging Plate Reader (FLIPR) uses an argon laser to rapidly scan a microtiter plate containing dye loaded cells and a semi-confocal detection method. Ca²⁺ levels are measured using indicators such as fluo-3 or Calcium Green, which are efficiently excited at 488 nm wavelength of the argon laser.

Patch clamping is another technique which allows for assessment of ionic currents at the level of the whole cell membrane. A freshly made glass pipette with a tip diameter of only a few micrometers (μm) is pressed gently on the cell membrane to form a gigaseal. When suction is applied to the pipette the membrane breaks and the cytoplasm and pipette solution containing a specific agent start to mix. This is all done by monitoring the voltage changes across a membrane. As ions move from one side of the membrane to another a particular voltage is produced. In effect, the researcher can determine when ions are moving by monitoring the voltage changes.

The instant methods will find application in the treatment of a wide range of conditions associated with abnormal mechanoreceptor activation. In a particularly contemplated aspect, the present methods will be useful where the subject has a disorder associated with either an excessive or inadequate dietary intake, including, without being limited to, obesity, anorexia, bulimia, diabetes and energy imbalance, sleep apnoea, neural injury, neurological diseases, severe burns, severe trauma, chronic non-neurological diseases, chronic infections, chronic corticosteroid administration, AIDS, and the like. Neural injuries include acute brain injuries, traumatic brain injuries, closed head injuries, stroke, and the like. Neurological diseases include chronic neurodegenerative diseases such as Alzheimer's disease, Parkinson's disease, Huntington's disease, multiple sclerosis, and the like.

As indicated above, the preferred ion channel acting as a mechanoreceptor is TRPV2. Accordingly, the present invention provides therapeutic compounds which are useful in the treatment or prophylaxis or modulation including control of obesity, anorexia, satiation, weight maintenance, metabolic energy levels, and/or inflammatory diseases wherein the preferred compounds are selected from a calcium uptake inhibitor or promoter, a blocker or promoter of mechanoreceptor TRPV2 calcium channels and a biological dye which inhibits or promotes calcium ion flux. Calcium flux needs calcium ion uptake and release, and in particular, the movement of calcium ions into and out of the cells of the stomach wall. Particularly preferred cells are neuronal cells of the myenteric plexus. More particularly, the compounds are selected from 1-[β-[3-(4-methoxyphenyl)propoxy]-4-methoxyphenethyl]-1H-imidazole, a ruthenium red dye and salts, homologs, orthologs, analogs, isomers, enantiomers derivatives or functional equivalents thereof. Particularly preferred ruthenium dyes include ruthenium(6+), tetradecaamminedi-m-oxotri-, hexachloride, trans-(8Cl) or a stereoisomer or enantiomer thereof and/or ammoniated ruthenium oxychloride or a stereoisomer or enantiomer thereof. The present invention further provides pharmaceutical compositions and methods of treatment and/or prophylaxis. Although the present invention is particularly directed to TRPV2, it also extends to TRPV1 and TRPV3 through TRPV6.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease. Generally, such a condition or disorder involves either an excessive intake of food or a deficiency in food intake.

A “subject” as used herein refers to an animal including an avian species, preferably a mammal and more preferably human who can benefit from the pharmaceutical formulations and methods of the present invention. Other terms such as recipient, patient, host or target may be used in place of subject. There is no limitation on the type of animal that could benefit from the presently described pharmaceutical formulations and methods. A subject regardless of whether a human or non-human animal may be referred to as an individual, subject, animal, host or recipient. The compounds and methods of the present invention have applications in human medicine, veterinary medicine as well as in general, domestic or wild animal husbandry. For convenience, an “animal” includes an avian species such as a poultry bird, an aviary bird or game bird.

The treatment of diseases and disorders associated with inappropriate food intake are also contemplated by the methods of the present invention.

The preferred animals are humans or other primates, livestock animals, laboratory test animals, companion animals or captive wild animals.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species, zebrafish and amphibians (including cane toads) are also contemplated.

Accordingly, in a preferred embodiment the present invention provides compounds which modulate calcium uptake thereby influencing factors involved in obesity, anorexia, satiation, weight maintenance, metabolic energy levels, and/or inflammatory diseases. Obesity and anorexia are described with reference to a subject being lean or obese.

The terms “lean” and “obese” are used in their most general sense but should be considered relative to the standard criteria for determining obesity. Generally, for human subjects the definition of obesity is BMI>30 kg/m² (Risk Factor Prevalence Study Management Committee. Risk Factor Prevalence Study: Survey No. 3:1989. Canberra: National hearth Foundation of Australia and Australian Institute of Health, 1990; Waters and Bennett, Risk Factors for Cardiovascular Disease: A Summary of Australian data. Canberra: Australian Institute of Health and Welfare, 1995).

Conveniently, an animal model may be employed to study the effects of obesity. In particular, PCT/AU02/01405 exemplifies the Psammomys obesus (the Israeli sand rat) animal model of dietary-induced obesity. In its natural desert habitat, an active lifestyle and saltbush diet ensure that they remain lean and normoglycemic. However, in a laboratory setting on a diet of ad libitum chow (on which many other animal species remain healthy), a range of pathophysiological responses are seen (Barnett et al., Diabetologia 37: 671-676, 1994a, Barnett et al., Int. J. Obesity 18: 789-794, 1994b, Barnett et al., Diabete Nutr Metab 8: 42-47, 1995). By the age of 16 weeks, more than half of the animals become obese and approximately one third develop diabetes and the most hyperphagic animals go on to develop hyperglycemia, highlighting the importance of excessive energy intake in the pathophysiology of obesity and diabetes in Psammomys obesus (Collier et al., Ann New York Acad Sci 827: 50-63, 1997a, Walder et al., Obesity Res 5: 193-200, 1997a; Collier et al., Exp Clin Endocrinol Diabetes 105: 36-37, 1997b).

Psammomys obesus animals are conveniently divided into three groups viz Group A animals which are lean, normoglycemic and normoinsulinemic, Group B animals which are obese, normoglycemic and hyperinuslinemic and Group C animals which are obese, hyperglycemic and hyperinsulinemic.

The present invention extends, however, to the targeting of calcium uptake in other test animals such as primates, livestock animals, laboratory test animals, companion animals or captive wild animals.

Examples of laboratory test animals include mice, rats, rabbits, guinea pigs and hamsters. Rabbits and rodent animals, such as rats and mice, provide a convenient test system or animal model. Livestock animals include sheep, cows, pigs, goats, horses, donkeys and canalids. Non-mammalian animals such as zebrafish and amphibians (including cane toads) may also be useful models.

The compounds of the present invention may be manufactured and/or used in preparation, i.e. manufacture or formulation or a composition such as a medicament, pharmaceutical composition or drug. These may be administered to individuals in a method of treatment or prophylaxis. Alternatively, they may be incorporated into a patch, slow release capsule or implant or stent or other device inserted into vessels or tissue such as a catheter.

Thus, the present invention extends, therefore, to a pharmaceutical composition, medicament, drug or other composition including a stent, catheter, patch or rapid or slow release formulation comprising agent selected from a calcium uptake inhibitor or promoter, a blocker or promoter of TRPV2 calcium channels and a biological dye which inhibits or promotes calcium uptake. In a preferred embodiment, the composition comprises 1-[β-[3-(4-methoxyphenyl)propoxy]-4-methoxyphenethyl]-1H-imidazol and/or a ruthenium red dye and/or salts, homologs, orthologs, analogs, isomers, enantiomers, derivatives or functional equivalents thereof as well as pharmaceutical compositions comprising same. Particularly preferred ruthenium dyes include ruthenium(6+), tetradecaamminedi-m-oxotrihexachloride, trans-(8Cl) or a stereoisomer or enantiomer thereof and/or ammoniated ruthenium oxychloride or a stereoisomer or enantiomer thereof. In addition, the pharmaceutical composition may further contain other agent(s) for use in controlling obesity, anorexia, satiation, weight maintenance, metabolic energy levels, and/or inflammatory diseases or the other agent(s) may be in a separate composition. Another aspect of the present invention contemplates a method comprising administration of such a composition to a patient such as for treatment or prophylaxis of an event or condition associated with obesity, anorexia, satiation, weight maintenance, metabolic energy levels, and/or inflammatory diseases. The compounds of the present invention may also be used in the manufacture of a medicament for the treatment or prophylaxis of an event or condition associated with obesity, anorexia, satiation, weight maintenance, metabolic energy levels and inflammatory conditions. Furthermore, the present invention contemplates a method of making a pharmaceutical composition comprising admixing a compound of the present invention with a pharmaceutically acceptable excipient, vehicle or carrier, and optionally other ingredients. Where multiple compositions are provided then such compositions may be given simultaneously or sequentially. Sequential administration includes administration within nanoseconds, seconds, minutes, hours or days. Preferably, within seconds or minutes.

Such compositions are proposed to be useful in the treatment and/or prophylaxis and/or control of obesity, anorexia, satiation, weight maintenance, metabolic energy levels, and/or inflammatory diseases.

Examples of metabolic diseases include various manifestations such as diabetes and disorders associated with imbalances in metabolic energy levels. Diseases and disorders associated with genetic disorders are also contemplated by the present invention.

Examples of inflammatory disease conditions contemplated by the present invention include but are not limited to those diseases and disorders which result in a response of redness, swelling, pain, and a feeling of heat in certain areas that is meant to protect tissues affected by injury or disease. Inflammatory diseases which can be treated using the methods of the present invention, include, without being limited to, acne, angina, arthritis, aspiration pneumonia, empyema, gastroenteritis, inflammation, intestinal flu, necrotizing enterocolitis, pelvic inflammatory disease, pharyngitis, pleurisy, raw throat, rubor, sore throat, stomach flu and urinary tract infections, Chronic Inflammatory Demyelinating Polyneuropathy, Chronic Inflammatory Demyelinating Polyradiculoneuropathy, Chronic Inflammatory Demyelinating Polyneuropathy and Chronic Inflammatory Demyelinating Polyradiculoneuropathy.

Accordingly, another aspect of the present invention contemplates a method for the treatment or prophylaxis of a condition in an animal, said method comprising administering to said animal an effective amount of a compound which inhibits or promotes calcium uptake as described herein or a composition comprising same.

Preferably, the animal is a mammal such as a human or is a laboratory test animal such as a mouse, rat, rabbit, guinea pig, hamster, zebrafish or amphibian.

Agents which modulate the activation of a mechanoreceptor may also be identified by assessing the ability of potential agents to activate or decrease or prevent activation of a mechanoreceptor. Such agents may be identified in natural product collections, combinatorial, synthetic/peptide polypeptide or protein libraries or using phage display or SELEX technology. A vast range of screening methods and high through put screening methods are available.

The target polypeptide or fragment employed in such a test may either be free in solution, affixed to a solid support, or borne on a cell surface. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may measure, for example, the formation of complexes between a target or fragment and the agent being tested, or examine the degree to which the formation of a complex between a target or fragment and a known ligand is aided or interfered with by the agent being tested.

The screening procedure includes assaying (i) for the presence of a complex between the drug and the target, or (ii) an alteration in the expression levels of nucleic acid molecules encoding the target. One form of assay involves competitive binding assays. In such competitive binding assays, the target is typically labeled. Free target is separated from any putative complex and the amount of free (i.e. uncomplexed) label is a measure of the binding of the agent being tested to target molecule. One may also measure the amount of bound, rather than free, target. It is also possible to label the compound rather than the target and to measure the amount of compound binding to target in the presence and in the absence of the drug being tested.

Another technique for drug screening provides high throughput screening for compounds having suitable binding affinity to a target and is described in detail in Geysen (International Patent Publication No. WO 84/03564). Briefly stated, large numbers of different small peptide test compounds are synthesized on a solid substrate, such as plastic pins or some other surface. The peptide test compounds are reacted with a target and washed. Bound target molecule is then detected by methods well known in the art. This method may be adapted for screening for non-peptide, chemical entities. This aspect, therefore, extends to combinatorial approaches to screening for target antagonists or agonists.

Purified target can be coated directly onto plates for use in the aforementioned drug screening techniques. However, non-neutralizing antibodies to the target may also be used to immobilize the target on the solid phase. The target may alternatively be expressed as a fusion protein with a tag conveniently chosen to facilite binding and identification.

Such agents may be identified and isolated as a result of screening programs or they may be developed based on the I-D, 2-D or 3-D structure of a mechanoreceptor or a molecule which binds to a mechanoreceptor together with tests as herein described.

Following identification of a suitable agent, it may be manufactured and/or used in a preparation, i.e. in the manufacture or formulation or a composition such as a medicament, pharmaceutical composition or drug. These may be administered to individuals in a method of treatment or prophylaxis. Alternatively, they may be incorporated into a patch or slow release capsule or implant.

The terms “compound”, “active agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The term “compound” is not to be construed as a chemical compound only but extends to peptides, polypeptides and proteins as well as genetic molecules such as RNA, DNA and chemical analogs thereof.

Thus, the present invention extends, therefore, to a pharmaceutical composition, medicament, drug or other composition including a patch or slow release formulation comprising an agent of the present invention. The present invention also provides dietary compositions for supplementing food or water supplies for companion livestock or wild life animal population to induce or suppress appetites.

Furthermore, the present invention contemplates a method of making a pharmaceutical or agricultural composition comprising admixing a compound of the instant invention with a pharmaceutically acceptable excipient, vehicle or carrier, and optionally other ingredients. Where multiple compositions are provided, then such compositions may be given simultaneously or sequentially. Sequential administration includes administration within nanoseconds, seconds, minutes, hours or days, preferably, within seconds or minutes.

Insofar as the agent is a genetic molecule, means is required to introduce a genetic molecule which is either an agent itself or encodes an agent into a target cell. Genetic molecule transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al., J. Gen. Virol. 73: 1533-1536, 1992), adenovirus (Berkner, Curr. Top. Microbiol. Immunol. 158: 39-66, 1992; Berkner et al., BioTechniques 6; 616-629, 1988; Gorziglia and Kapikian, J. Virol. 66: 4407-4412, 1992; Quantin et al., Proc. Natl. Acad. Sci. USA 89: 2581-2584, 1992; Rosenfeld et al., Cell 68: 143-155, 1992; Wilkinson et al., Nucleic Acids Res. 20: 2233-2239, 1992; Stratford-Perricaudet et al., Hum. Gene Ther. 1: 241-256, 1990; Schneider et al., Nature Genetics 18: 180-183, 1998), vaccinia virus (Moss, Curr. Top. Microbiol. Immunol. 158: 25-38, 1992; Moss, Proc. Natl. Acad. Sci. USA 93: 11341-11348, 1996), adeno-associated virus (Muzyczka, Curr. Top. Microbiol. Immunol. 158: 97-129, 1992; Ohi et al., Gene 89: 279-282, 1990; Russell and Hirata, Nature Genetics 18: 323-328, 1998), herpesviruses including HSV and EBV (Margolskee, Curr. Top., Microbiol. Immunol. 158: 67-95, 1992; Johnson et al., J. Virol. 66: 2952-2965, 1992; Fink et al., Hum. Gene Ther. 3: 11-19, 1992; Breakefield and Geller, Mol. Neurobiol. 1: 339-371, 1987; Freese et al., Biochem. Pharmacol. 40: 2189-2199, 1990; Fink et al., Ann. Rev. Neurosci. 19: 265-287, 1996), lentiviruses (Naldini et al., Science 272: 263-267, 1996), Sindbis and Semliki Forest virus (Berglund et al., Biotechnology 11: 916-920, 1993) and retroviruses of avian (Bandyopadhyay and Temin, Mol. Cell. Biol. 4: 749-754, 1984; Petropoulos et al., J. Viol. 66: 3391-3397, 1992], murine [Miller, Curr. Top. Microbiol. Immunol. 158: 1-24, 1992; Miller et al., Mol. Cell. Biol. 5: 431-437, 1985; Sorge et al., Mol. Cell. Biol. 4: 1730-1737, 1984; Mann and Baltimore, J. Virol. 54: 401-407, 1985; Miller et al., J. Virol. 62: 4337-4345, 1988] and human [Shimada et al., J. Clin. Invest. 88: 1043-1047, 1991; Helseth et al., J. Virol. 64: 2416-2420, 1990; Page et al., J. Virol. 64: 5270-5276, 1990; Buchschacher and Panganiban, J. Virol. 66: 2731-2739, 1982] origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, amide, prodrug or derivative of a compound as provided herein is a salt, ester, amide, prodrug or derivative that this not biologically or otherwise undesirable.

Liposome/DNA complexes have been shown to be capable of mediating direct in vivo genetic molecule transfer. While in standard liposome preparations the gene transfer process is non-specific, localized in vivo uptake and expression have been reported in tumor deposits, for example, following direct in situ administration.

If the genetic molecule encodes a sense or antisense polynucleotide or a ribozyme or DNAzyme, expression will produce the sense or antisense polynucleotide or ribozyme or DNAzyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

Agents are formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. topical, intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is unacceptably toxic or if it would otherwise require too high a dosage or if it would not otherwise be able to enter the target cells.

Instead of administering these agents directly, they could be produced in the target cell, e.g. in a viral vector such as described above or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

The present invention is further described by the following non-limiting Examples.

### EXAMPLE 1

### Identification of Mechanoreceptors Associated with Gastric Distension

Table 3 describes 33 genes which are associated with gastric distension.

TABLE 3
Mechanoreceptors for gastric distension

| ION | | GENE | RAT | | |
|---|---|---|---|---|---|
| CHANNEL | CHR. | NAME | HOMOLOG | HOMOLOGY | REFSEQ |
| αENAC | 12p13 | SCNN1A | Scnn1a | 83 | NM_031548.1 |
| βENAC | 16p12 | SCNN1B | Scnn1b | 84 | NM_012648.1 |
| γENAC | 16p12 | SCNN1C | Scnn1c | 84 | NM_017046.1 |
| δENAC | 1p36 | SCNN1D | — | — | — |
| ASIC1 | 12q12 | ACCN2 | Accn2 | 89 | NM_024154.1 |
| ASIC2 | 17q11 | ACCN1 | Accn1 | 89 | NM_012892.1 |
| ASIC3 | 7q35 | ACCN3 | Asic3 | 82 | NM_173135.1 |
| ASIC4 | 2q36 | ACCN4 | Asic4 | 85 | NM_022234.1 |
| BLINAC/hiNaC | 4q31 | ACCN5 | Inac | 83 | NM_022227 |
| TREK1 | 1q41 | KCNK2 | Kcnk2 | 86 | NM_172042.1 |
| TREK2 | 14q31 | KCNK10 | Kcnk10 | 89 | NM_023096.2 |
| TRAAK | 11q13 | KCNK4 | LOC360318 | 83 | XM_346568.1 |
| TRPM1 | 15q13 | TRPM1 | LOC361586 | 88 | XM_341868.1 |
| TRPM2 | 21q22 | TRPM2 | LOC294329 | 83 | XM_228069.2 |
| TRPM3 | 9q21 | TRPM3 | LOC309407 | 91 | XM_219902.2 |
| TRPM4 | 19q13 | TRPM4 | Mls2s | 81 | NM_133607.1 |
| TRPM5 | 11p15 | TRPM5 | LOC365391 | 85 | XM_344979.1 |
| TRPM6 | 9q21 | TRPM6 | LOC293874 | 83 | XM_219747.2 |
| TRPM7 | 15q21 | TRPM7 | — | — | — |
| TRPM8 | 2q37 | TRPM8 | CMR1 | 87 | NM_134371.1 |
| TRPC1 | 3q23 | TRPC1 | LOC360438 | 84 | XM_346834.1 |
| TRPC2 | 11p15 | TRPC2 | Trpc2 | 85 | NM_022638.1 |
| TRPC3 | 4q27 | TRPC3 | Trpc3 | 85 | NM_021771.1 |
| TRPC4 | 13q13 | TRPC4 | Trpc4 | 87 | NM_080396.1 |
| TRPC5 | Xq23 | TRPC5 | LOC360455 | 90 | XM_346868.1 |
| TRPC6 | 11q21 | TRPC6 | Trrp6 | 89 | NM_053559.1 |
| TRPC7 | 5q31 | TRPC7 | LOC306757 | 91 | XM_225159.2 |
| TRPV1 | 17p13 | TRPV1 | Trpv1 | 85 | NM_031982.1 |
| TRPV2 | 17p11 | TRPV2 | Vr11 | 81 | NM_017207.1 |
| TRPV3 | 17p13 | TRPV3 | — | — | — |
| TRPV4 | 12q24 | TRPV4 | Trpv4 | 81 | NM_023970.1 |
| TRPV5 | 7q35 | TRPV5 | LOC360382 | 83 | XM_346696.1 |
| TRPV6 | 7q34 | TRPV6 | Trpv6 | 81 | NM_053686.1 |

### EXAMPLE 2

### Tissue Expression of Candidate Genes

Oligonucleotide primers suitable for polymerase chain reaction amplification were designed for the genes identified in Table 3. Primer sequences for each of the genes are listed in Table 4.

TABLE 4

Primer sequences for amplification
of candidate ion channels

| GENE | | |
|---|---|---|
| NAME | FORWARD PRIMER | REVERSE PRIMER |
| SCNN1A | GCCTGGGCTGTTTCTCCAA | CGTGAGTAGCCGGCAGAGAG |
| SCNN1B | GTGCAAAGTGGCCATGAGG | GCACTGGTGAAGTTTCGGAAG |
| SCNN1C | GCCAATCAGTGTGCAAGCAA | GAAGCCTCAGACGGCCATT |
| ACCN2 | GCCAACTTCCGGAGCTTCA | GGCACGAGAGCAGCATGTC |
| ACCN1 | CATCACAGCCTGTCGGATTG | GCAGGCTCTGCACACTCCTT |
| ACCN3 | CCCAGTCCGACCTTTGACA | TCGGCAATCCAACAACATGT |
| ACCN4 | AACCTGCTTCCCAACCATCAC | CTTTCCCCACACAGCACCAT |
| ACCN5 | CTGGGCTCTGTCTCGCTCTT | GTTGTCGTTGGCCACATGAA |
| KCNK2 | TGCCATAAGGCCTCTGAATGA | CTCAGTTAGGCGAACCCTGAA |
| KCNK10 | GCCGTTTCAAGGCCTCATC | TGAGCTGTTCTGGCCCCTTA |
| KCNK4 | GGAGCAAGCTGAAAGCCATCT | GGCTGGTAGGCTGGAGAGTTC |
| TRPM1 | CTGTCCCTGTGGTGGTTTGTG | CGTCGCAGTATTTGTGTGCAA |
| TRPM2 | GAGGAGACACGGCAGCTATTCT | TGAAGTACAGGGACGCCATCT |
| TRPM3 | TGGCTGCAGGAGTACTGGAA | CCTGAAGGGCTGGTCTTGAAG |
| TRPM4 | CAACAAAGTGCATGGCAACAG | GAATTCCCGGATGAGGCTG |
| TRPM5 | ATGGGAGCCAGTCCTATGCA | CCTTCACAAACTTGCTTCGCT |
| TRPM6 | TCTCAGCCACTGAGGGCAAT | GGAAGTTAATGGTGCCGAAGG |
| TRPM8 | CTGCTGGAGTGGAACCAACTG | AGGGCCGTGAACATGACTTC |
| TRPC1 | CAAAGCAACGACACCTTCCA | GCCACATGCGCTAAGGAGAA |
| TRPC2 | CCCTACCAGGAGACGGAGAAG | GCCAAACATGGTCCAGAAGAG |
| TRPC3 | GGGCAGGTGACGACTTCTATG | AGGATGATGGGCGTGATGTC |
| TRPC4 | CCACGAGGTCCGCTGTAACT | CGTGAGTGCCTGAGGCTGT |
| TRPC5 | CCTTCGCTCATCGCCTTATC | CCTTGAGTTCCCAGCCCAG |
| TRPC6 | GCAGCAGCTCCTCTCCATATG | CGAGGACCACGAGGAATTTC |
| TRPC7 | TCCTGGACGGAGATGCTCAT | TCCTCCCAGATCTCCTTGCA |
| TRPV1 | CAGCACTGCACATTGCCATT | TCCATTCTCCACCAAGAGGGT |
| TRPV2 | GGTCATCCTTCGAGACCTGC | GGCTACAGCAAAGCCGAAAA |
| TRPV4 | CTGATGAGGAGTTCCGGGAA | CGTTTCGGCCATTGCTTAAG |
| TRPV5 | GTTGCGAATATGGCCTGGG | TCGATACGGATTCTGCTCCTG |
| TRPV6 | TGCAGCCCAACAAAACCTTT | GGGCACAAGTTCAAGGGACTT |

The candidate ion channels were tested for expression in the stomach, as well as a range of other rat tissues. The tissues were obtained from a single male Sprague-Dawley rat (Rattus norvegicus) at 20 weeks of age. The animal was killed by pentobarbitone overdose (120 mg/kg) followed by cervical dislocation. The following tissues were rapidly excised and snap frozen in liquid nitrogen: spleen, pancreas, epididymal fat, stomach, colon and brain. Total RNA was extracted from the tissues using standard protocols, and PCR was performed to detect expression of each of the candidate ion channels in each of these tissues. The results of this experiment are detailed in Table 5.

TABLE 5
Tissue expression of candidate ion channels in rats

| | | PAN- | | | CO- | |
|---|---|---|---|---|---|---|
| GENE | SPLEEN | CREAS | FAT | STOMACH | LON | BRAIN |
| Scnn1a | ✓ | | ✓ | ✓ | ✓ | |
| Scnn1b | ✓ | | ✓ | ✓ | ✓ | ✓ |
| Scnn1c | ✓ | | ✓ | ✓ | ✓ | ✓ |
| Accn2 | ✓ | | ✓ | ✓ | ✓ | ✓ |
| Accn1 | ✓ | | ✓ | ✓ | ✓ | ✓ |
| Asic3 | ✓ | | ✓ | ✓ | ✓ | ✓ |
| Asic4 | ✓ | | ✓ | ✓ | ✓ | ✓ |
| Inac | ✓ | | ✓ | ✓ | ✓ | ✓ |
| Kcnk2 | ✓ | | ✓ | ✓ | ✓ | ✓ |
| Kcnk10 | ✓ | | ✓ | ✓ | ✓ | ✓ |
| LOC360318 | ✓ | | ✓ | ✓ | ✓ | ✓ |
| LOC361586 | ✓ | | ✓ | ✓ | ✓ | ✓ |
| LOC294329 | ✓ | | ✓ | ✓ | ✓ | ✓ |
| LOC309407 | ✓ | | ✓ | ✓ | ✓ | ✓ |
| Mls2s | ✓ | | ✓ | ✓ | ✓ | ✓ |
| LOC365391 | ✓ | | ✓ | | ✓ | ✓ |
| LOC293874 | ✓ | | ✓ | ✓ | ✓ | ✓ |
| CMR1 | ✓ | | ✓ | ✓ | ✓ | ✓ |
| LOC360438 | ✓ | | ✓ | ✓ | ✓ | ✓ |
| Trpc2 | ✓ | | ✓ | ✓ | ✓ | ✓ |
| Trpc3 | ✓ | | ✓ | ✓ | ✓ | ✓ |
| Trpc4 | ✓ | | ✓ | ✓ | ✓ | ✓ |
| LOC360455 | ✓ | | ✓ | | ✓ | ✓ |
| Trrp6 | ✓ | | ✓ | ✓ | ✓ | ✓ |
| LOC306757 | | | | | | ✓ |
| Trpv1 | ✓ | | ✓ | ✓ | ✓ | ✓ |
| Vr11 | ✓ | | ✓ | ✓ | ✓ | ✓ |
| Trpv4 | | | ✓ | | ✓ | ✓ |
| LOC360382 | | | | | | |
| Trpv6 | ✓ | | ✓ | ✓ | ✓ | ✓ |

Due to the fact that they are not expressed in the stomach, and therefore could not function in detection of gastric distension, the following ion channels were excluded from further investigation: TRPM5 (LOC365391), TRPC5 (LOC360455), TRPC7 (LOC360757), TRPV4 (Trpv4) and TRPV5 (LOC360382).

### EXAMPLE 3

### Expression of Candidate Mechanoreceptors in Fasted and Fed Animals

The remaining 25 candidate ion channels were tested for changes in expression after gastric distension in Sprague-Dawley rats that were fasted for 24 h or refed after fasting for 1 or 4 hours (n=5-6 in each group). Ion channel(s) activated by gastric distension demonstrate increased expression in the refed (distended stomach) compared with the fasted state (see Table 6).

TABLE 6
Expression of candidate ion channels in fasted and refed rats

| GENE | FASTED¹ | REFED 1 h¹ | REFED 4 h¹ | Max. change |
|---|---|---|---|---|
| SCNN1A | 11.3 ± 2.6 | 15.5 ± 2.7 | 17.2 ± 4.3 | ↑52% |
| SCNN1B | 15.5 ± 6.3 | 68.2 ± 18.9 | 53.0 ± 20.4 | ↑340% |
| SCNN1C | 8.7 ± 2.5 | 8.2 ± 1.6 | 6.9 ± 0.9 | ↓21% |
| ACCN2 | 11.2 ± 2.2 | 8.1 ± 2.2 | 21.8 ± 11.9 | ↑95% |
| ACCN1 | 13.0 ± 5.0 | 20.3 ± 3.1 | 22.7 ± 5.2 | ↑75% |
| ACCN3 | 30.1 ± 12.6 | 2.6 ± 0.6 | 3.6 ± 1.9 | ↓91% |
| ACCN4 | 12.8 ± 3.9 | 6.7 ± 1.8 | 6.5 ± 1.8 | ↓49% |
| ACCN5² | 32.2 ± 17.7 | 1.3 ± 0.3 | 0.7 ± SE | ↓98% |
| KCNK2 | 14.0 ± 4.2 | 10.1 ± 1.0 | 7.5 ± 1.1 | ↓46% |
| KNK10 | 10.5 ± 1.6 | 13.4 ± 5.8 | 12.4 ± 4.2 | ↑28% |
| KCNK4² | 11.4 ± 2.3 | 11.9 ± 3.0 | 10.6 ± 2.1 | ↓7% |
| TRPM1³ | 12.5 ± 3.4 | 8.8 ± 2.8 | 5.9 ± 2.1 | ↓53% |
| TRPM2 | 34.1 ± 18.3 | 2.7 ± 0.4 | 2.8 ± 0.7 | ↓92% |
| TRPM4³ | 11.1 ± 2.4 | 13.1 ± 1.5 | 7.2 ± 0.7 | ↓35% |
| TRPM6³ | 10.5 ± 1.5 | 12.8 ± 5.6 | 11.9 ± 4.0 | ↑22% |
| TRPM8 | 11.4 ± 2.4 | 11.8 ± 2.9 | 8.9 ± 1.8 | ↓22% |
| TRPC1 | 12.5 ± 3.4 | 8.7 ± 2.8 | 6.7 ± 2.4 | ↓46% |
| TRPC3 | 10.8 ± 1.8 | 8.1 ± 1.2 | 9.6 ± 1.6 | ↓25% |
| TRPC4 | 8.4 ± 2.5 | 8.9 ± 1.7 | 7.6 ± 2.3 | ↓10% |
| TRPC6 | 1.8 ± 0.4 | 1.4 ± 0.2 | 1.2 ± 0.1 | ↓50% |
| TRPV1 | 10.9 ± 2.1 | 8.0 ± 1.0 | 5.7 ± 1.1 | ↓48% |
| TRPV2² | 11.1 ± 2.4 | 4.8 ± 0.8 | 4.5 ± 0.8 | ↓59% |
| TRPV6³ | 13.4 ± 5.3 | 10.9 ± 2.1 | 10.6 ± 1.8 | ↓21% |

¹Mean ± SEM, arbitrary units
²Genes previously replicably linked/associated with obesity phenotypes
³Genes previously linked/associated with obesity phenotypes

As shown in Table 6, a number of putative mechanoreceptors for physiological gastric distension have been identified. Of particular interest, TRPV2 gene expression was reduced by 59% (p=0.031) after re-feeding, which induced gastric distension in the rats. ACCN5 gene expression was reduced by 98% after re-feeding, its expression was virtually abolished when the stomach was distended. Both of these genes are located in regions replicably linked with obesity phenotypes, and in conjunction with this gene expression data this makes them excellent candidates for signalling physiological gastric distension and playing a role in the sensation of satiety.

A number of other genes showed differential expression after gastric distension, and are also associated with the sensation of satiety perception. These include TRPM1, TRPM4 and TRPV6 (expression reduced by 53, 35 and 21%, respectively, after re-feeding) and TRPM6 (expression increased by 22% after re-feeding). All of these genes are located in genomic regions previously linked or associated with obesity phenotypes. These genes are therefore strong candidates for the mechanotransduction of gastric distension, and may play a role in the sensation of satiety.

Of the remaining genes many demonstrated increased gene expression after re-feeding (ranging from 28-340% increase), while a number of other genes exhibited decreased expression after re-feeding (ranging from 7-92% decrease). These genes that show a substantial change in expression following gastric distension are also involved in the sensation of satiety.

As shown above, a number of ion channels that show altered expression following gastric distension have been identified, demonstrating that they are regulated in response to this mechanical stimulus. Each of these ion channels is a potential target for development of obesity treatments. Specifically, as stated above, the identification of chemical compounds that can activate or block these ion channels independent of mechanical stimuli could induce a sensation of satiety despite the fact that the stomach is not distended, and therefore could be useful in the treatment of obesity.

### EXAMPLE 4

### Genomic Location and Expression Profiles of Candidate Mechanoreceptor Genes

Many of these ion channels are of particular interest as they are located in genomic regions previously linked with obesity. This provides further evidence that they may be causally involved in the pathophysiology of the disease.

Particularly, TRPV2 is located on chromosome 17p11 in a region strongly linked with plasma leptin concentrations (LOD score=4.97) in Caucasians in the USA (Kissebah A H, et al. Proc Natl Acad Sci USA. 97: 14478-83, 2000). This same region was linked with body mass index (BMI), another obesity phenotype, with a maximum LOD score of 2.47, in a combined analysis of white, black, Mexican and Asian Americans (Wu X, et al. Am J Hum Genet. 70: 1247-56, 2002). Taken together, these studies provide convincing evidence that either TRPV2 or a nearby gene plays a significant causal role in the development of obesity.

TRPV4 is located on chromosome 12q24. This genomic region was linked with BMI in Finnish and Swedish Caucasians (LOD score=1.85) (Parker A, et al. Diabetes 50: 675-80, 2001), and associated with BMI in a Caucasian population (p=0.03) (Acton S, et al. Arterioscler Thromb Vasc Biol. 19: 1734-43, 1999). Furthermore, in the Quebec Family Study, this region of chromosome 12 was linked with waist circumference (LOD score=2.88), another obesity phenotype (Perusse L, et al. Diabetes 50: 614-21, 2001). Taken together, these studies provide convincing evidence that either TRPV4 or a nearby gene plays a significant causal role in the development of obesity.

TRPM5 and TRPC2 are both located on chromosome 11p15 in a region linked with obesity in Mexican Americans (LOD score=1.6) (Duggirala R, et al. Am J Hum Genet. 68: 1149-64, 2001), and associated with BMI in French Caucasians (p=0.0032) (Hani E H, et al. Diabetes 46: 688-94, 1997). Therefore it is highly likely that TRPM5 or TRPC2 or a nearby gene contribute to the development of obesity.

KCNK4 (TRAAK) is located on chromosome 11q13. This region was associated with obesity (p=0.006) in the Quebec Family Study (Bouchard C, et al. Hum Mol Genet. 6: 1887-9, 1997), and showed evidence of linkage with BMI (LOD score=2.2) in study in Caucasian subjects (Chagnon Y C, et al. J Appl Physiol. 90: 1777-87, 2001). These studies support the contention that KCNK4 or a nearby gene plays a causal role in the development of obesity.

ACCN5 (BLINAC/hiNaC) is located on chromosome 4q31 in a region linked to BMI in both French Caucasians (LOD score=2.09) (Vionnet N, et al. Am J Hum Genet. 67: 1470-80, 2000) and Ashkenazi Jews (LOD score=2.41) (Permutt M A, et al. Diabetes 50: 681-5, 2001). Therefore it is highly likely that ACCN5 or a nearby gene contributes to the development of obesity.

TRPV6 is located on chromosome 7q34, a region strongly linked with BMI (LOD score=3.8) in the National Heart, Lung, and Blood Institute Family Heart Study (Feitosa M F, et al. Am J Hum Genet. 70: 72-82, 2002). TRPM1 is located on chromosome 15q13, a region also linked with BMI (LOD score=1.6) in the National Heart, Lung, and Blood Institute Family Heart Study (Feitosa M F, et al. Am J Hum Genet. 70: 72-82, 2002). TRPM4 is located on chromosome 19q13, a region strongly linked with BMI (LOD score=2.6) in Mexican Americans (Arya R, et al. Nat Genet. 30: 102-5, 2002). Finally, TRPM3 and TRPM6 are both located on chromosome 9q21, a region linked with BMI (LOD score=1.7) in the Framingham population (Atwood L D, et al. Am J Hum Genet. 71: 1044-50, 2002). Therefore, there is some evidence that TRPV6, TRPM1, TRPM4, TRPM3 or TRPM6 could be involved in the development of obesity in a range of populations.

### EXAMPLE 5

### 1-[β[3-(4-methoxyphenyl)propoxy]-4-methoxyphenethyl]-1H-imidazole

This compound is available from BIOMOL Research Laboratories (PA, USA) under catalog number CA-230 and product identification SK&F 96365.

It is a white solid with a melting point of 117-119° C. Its molecular formula is C₂₂H₂₆N₂O₃.HCl and it has a molecular weight of 402.9. Solubility in water is 30 mg/ml.

This compound is a selective inhibitor of receptor-mediated Ca²⁺ entry in stimulated platelets (IC₅₀=8-12 μM), neutorphils and endothelial cells.

### EXAMPLE 6

### Ruthenium Red

This compound is a biological dye and general inhibitor of calcium uptake. It blocks all TRPV channels. Its chemical formula is H₄₂C₁₆N₁₄O²Ru₃4H₂O. It is available from Electron Microscopy Sciences (PA, USA) under Catalog Number 20600.

### EXAMPLE 7

### Intragastric gavage of 1-[beta-[3-(4-Methoxyphenyl)propoxy]-4-methoxyphenethyl]-1H-imidazole.HCl inhibits food intake in rats

Adult, male rats were fasted for 16 hours then administered with a single intragastric gavage of 1-[beta-[3-(4-Methoxyphenyl)propoxy]-4-methoxyphenethyl]-1H-imidazole.HCl at a dose of 5 mg/kg in saline in a volume of 0.5 ml, or saline alone (0.5 ml). There were 8 animals in each group. Food intake was measured at various timepoints over the following 24 hours. As shown in Table 7, 1-[beta-[3-(4-Methoxyphenyl)propoxy]-4-methoxyphenethyl]-1H-imidazole.HCl administration reduced food intake over the period of the study, with statistically significant effects after 2 h, 7 h, 11 h and 12 h (p<0.05, independent samples t-test). There were strong trends for reduced food intake after 3 h, 5 h, 6 h, 8 h, 9 h, 10 h and 24 h (p<0.10). Taken together, these data strongly suggest that oral administration of 1-[beta-[3-(4-Methoxyphenyl)propoxy]-4-methoxyphenethyl]-1H-imidazole.HCl reduces food intake in rats, and therefore this compound has potential use as an orally administered regulator of food intake in humans.

TABLE 7
Food Intake

| | Food Intake (g)* | | |
|---|---|---|---|
| Time (h) | Treated (n = 8) | Control (n = 8) | p-value |
| 0.5 | 2.4 ± 0.3 | 2.9 ± 0.2 | 0.175 |
| 1 | 2.8 ± 0.3 | 3.2 ± 0.3 | 0.295 |
| 2 | 2.9 ± 0.3 | 4.0 ± 0.3 | 0.038 |
| 3 | 3.8 ± 0.4 | 4.9 ± 0.3 | 0.062 |
| 4 | 4.9 ± 0.5 | 5.6 ± 0.3 | 0.315 |
| 5 | 5.1 ± 0.5 | 6.6 ± 0.3 | 0.055 |
| 6 | 6.3 ± 0.5 | 7.5 ± 0.3 | 0.078 |
| 7 | 7.4 ± 0.6 | 8.9 ± 0.3 | 0.035 |
| 8 | 8.2 ± 0.6 | 9.7 ± 0.3 | 0.054 |
| 9 | 9.3 ± 0.6 | 10.8 ± 0.3 | 0.093 |
| 10 | 10.3 ± 0.6 | 11.7 ± 0.3 | 0.078 |
| 11 | 11.5 ± 0.6 | 13.7 ± 0.3 | 0.021 |
| 12 | 12.4 ± 0.6 | 14.7 ± 0.3 | 0.011 |
| 24 | 18.4 ± 0.8 | 20.1 ± 0.3 | 0.087 |

*mean ± sem.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of said steps or features.

### BIBLIOGRAPHY

Acton, et al. Arterioscler Thromb Vasc Biol. 19: 1734-43, 1999
Arya, et al. Nat Genet. 30: 102-5, 2002
Atwood, et al. Am J Hum Genet. 71: 1044-50, 2002
Australian Institute of Health and Welfare (AIWH), Australia's Health, 2002, Canberra: AIWH
Barnett et al., Diabetologia 37: 671-676, 1994a
Barnett et al., Diabete Nutr Metab 8: 42-47, 1995
Barnett et al., Int. J. Obesity 18: 789-794, 1994b
Berthoud, et al. Am J Physiol 280: R1371-R1381, 2001
Bouchard, The genetics of Obesity, Boca Raton: CRC Press, 1994
Bouchard, et al. Hum Mol Genet. 6: 1887-9, 1997
Chagnon, et al. J Appl Physiol. 90: 1777-87, 2001
Chang, et al. Science 282: 2220-2226, 1998
Collier et al., Ann New York Acad Sci 827: 50-63, 1997a
Collier et al., Exp Clin Endocrinol Diabetes 105: 36-37, 1997b
Duggirala, et al. Am J Hum Genet. 68: 1149-64, 2001
Dunstan et al., Diabetes Res. Clin. Pract. 57: 119-129, 2002
Feitosa, et al. Am J Hum Genet. 70: 72-82, 2002
Fox, et al. J Neuroscience 21: 8602-8615, 2001
Garcia-Anoveros J, Corey D P. Annu Rev Neurosci 20: 567-594, 1997
Garcia-Anoveros J, et al. Proc Natl Acad Sci USA 94: 1459-1464, 1997
Gillespie et al, Nature 413: 194-202, 2001
Hamill et al, Physiol Rev 81: 685-740, 2001
Hani, et al. Diabetes 46: 688-94, 1997
Kellenberger et al, Physiol Rev 82: 735-767, 2002
Kissebah, et al. Proc Natl Acad Sci USA. 97: 14478-83, 2000
Kopelman, Nature 404: 635-643, 2000
Marx, Science 686-689, 2002
Mokdad et al., JAMA 282(16): 1519-1522, 1999
Must et al., JAMA 282(16): 1523-1529, 1999
National Health and Medical Research Council, Acting on Australia's weight: A strategy for the prevention of overweight and obesity. Canberra: National Health and Medical Research Council, 1996
Ozaki, et al. J Neurophysiol 82: 2210-2220, 1999
Ozaki, Gebhart G F. Am J Physiol 281: G1449-G1459, 2001
Parker, et al. Diabetes 50: 675-80, 2001
Patel, et al. Curr Opin Cell Biol 13: 422-427, 2001
Permutt, et al. Diabetes 50: 681-5, 2001
Perusse, et al. Diabetes 50: 614-21, 2001
Phillips and Powley, Am J Physiol 274: R1626-R1638, 1998
Price, et al. Nature 407: 1007-1011, 2000
Risk Factor Prevalence Study Management Committee. Risk Factor Prevalence Study: Survey No. 3:1989. Canberra: National hearth Foundation of Australia and Australian Institute of Health, 1990
Shin, et al. Nature Neuroscience 6: 724-730, 2003
Sidi, et al. Science 301: 96-99, 2003
Vionnet, et al. Am J Hum Genet. 67: 1470-80, 2000
Walder et al., Obesity Res 5: 193-200, 1997a
Walker, et al. Science 287: 2229-2234, 2000
Waters and Bennett, Risk Factors for Cardiovascular Disease: A Summary of Australian data. Canberra: Australian Institute of Health and Welfare, 1995
World Trade Organisation. Obesity. Preventing and managing the global epidemic. Report of a WHO Consultation on Obesity. Geneva: World Health Organisation, 1998\
Wu, et al. Am J Hum Genet. 70: 1247-56, 2002
Zagorodnyuk, et al. J Physiol 534: 255-268, 2001
Zhou, et al. Proc Natl Acad Sci USA 100: 7105-7110, 2003
Zimmet, Diabetes Care 15: 232-252, 1992

1-35. (canceled)

36. A method for modulating the perception of satiety in a subject, said method comprising administering to said subject an effective amount of an agent selected from the group consisting of an agonist of a mechanoreceptor, an antagonist of a mechanoreceptor, an inhibitor of expression of a gene encoding a mechanoreceptor and an enhancer of the expression of a gene encoding a mechanoreceptor wherein said agent changes the perception of satiety in said subject.

37. The method of claim 36, wherein said mechanoreceptors are selected from the group consisting of ENAC, βENAC, γENAC, ACCN3, ACCN4, ASIC 1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV2, TRPV3, TRPV6 and TRPM8.

38. The method of claim 37, wherein said mechanoreptors are selected from the group consisting of TRPV2, ACCN5, TRPM1, TRPM4, TRPV6 and TRPV4.

39. The method of claim 38, wherein said mechanoreceptor is TRPV2.

40. The method of claim 36, wherein said agonist of a mechanoreceptor promotes the perception of satiety.

41. The method of claim 36, wherein said antagonist of a mechanoreceptor reduces the perception of satiety.

42. The method of claim 41, wherein said antagonist is of TRPV2.

43. The method of claim 42, wherein said antagonist of TRPV2 is selected from the group consisting of 1-[β-[3-(4-methoxyphenyl)propoxy]-4-methoxyphenethyl]-1H-imidazole, ruthenium red dye and salts, homologs, orthologs, analogs, isomers, enantiomers, derivatives and functional equivalents thereof.

44. The method of claim 43, wherein said ruthenium red dye is selected from the group consisting of ruthenium(6t), tetradecaaminedi-m-oxotrihexachloride, a trans(8Cl) isomer of tetradecaaminedi-m-oxotrihexachloride, an enantiomer of tetradecaaminedi-m-oxotrihexachloride, an ammoniated ruthenium oxychloride, a steroisomer of ammoniated ruthenium oxychloride and an enantiomer of ammoniated ruthenium oxychloride.

45. The method of claim 36, wherein the subject is selected from the group consisting of a mammal.

46. The method of claim 45, wherein said mammal is selected from the group consisting of a primate, a human and a laboratory test animal.

47. A composition used to modulate the perception of satiety in a subject, said composition selected from the group consisting of an agonist of a mechanoreceptor, an antagonist of a mechanoreceptor, an inhibitor of the expression of a gene encoding a mechanoreceptor and an enhancer of the expression of a gene encoding a mechanoreceptor and a pharmaceutically acceptable carrier or diluent.

48. The composition of claim 47, wherein said mechanoreceptors are selected from the group consisting of ENAC, βENAC, γENAC, ACCN3, ACCN4, ASIC 1, ASIC2, ASIC3, ASIC4, BLINAC/hiNaC (ACCN5), TREK1, TREK2, TRAAK (KCNK4), SCNN1C, KCNK2, TRPM1, TRPM2, TRPM3, TRPM4, TRPM6, TRPM7, TRPM8, TRPC1, TRPC2, TRPC3, TRPC4, TRPC6, TRPV2, TRPV3, TRPV6 and TRPM8.

49. The composition of claim 48, wherein said antagonist is of TRPV2.

50. The composition of claim 49, wherein said antagonist of TRPV2 is selected from the group consisting of 1-[β-[3-(4-methoxyphenyl)propoxy]-4-methoxyphenethyl]-1H-imidazole, ruthenium red dye and salts, homologs, orthologs, analogs, isomers, enantiomers, derivatives and functional equivalents thereof.

51. The composition of claim 50, wherein said ruthenium red dye is selected from the group consisting of ruthenium(6t), tetradecaaminedi-m-oxotrihexachloride, a trans(8Cl) isomer of tetradecaaminedi-m-oxotrihexachloride, an enantiomer of tetradecaaminedi-m-oxotrihexachloride, an ammoniated ruthenium oxychloride, a steroisomer of ammoniated ruthenium oxychloride and an enantiomer of ammoniated ruthenium oxychloride.

52. A method comprising;
administering to an animal an effective amount of a compound selected from the group consisting of a calcium uptake inhibitor, a calcium uptake promoter, a blocker of TRPV2 calcium channels and a promoter of TRPV2 calcium channels and a biological dye which alters calcium uptake in order to ameliorate one or more symptoms from a disease selected from the group consisting of obesity, anorexia, need of satiation, weight maintenance conditions, metabolic energy levels and inflammatory disease.

53. The method of claim 52, wherein said alteration of calcium uptake is selected from the group consisting of an inhibition and a promotion.

54. The method of claim 52, wherein said the compound is selected from the group consisting of 1-[β-[3-(4-methoxyphenyl)propoxy]-4-methoxyphenethyl]-1H-imidazole, a ruthenium red dye and salt, homolog, ortholog, analog, isomer, enantiomer, derivative or functional equivalent thereof.

55. The method of claim 52, wherein said animal is a mammal.

56. The method of claim 52, wherein said compounds modulate calcium ion uptake in cells of the stomach wall.

57. The method of claim 56, wherein said cells are neuronal cells of the myenteric plexus.
